(19) United States
(12) Patent Application Publication
(10) Pub. No.: US 2019/0204312 A1
(43) Pub. Date: Jul. 4, 2019

(54) Magnetic particle-binding peptides

(71) Applicants: TECHNISCHE UNIVERSITÄT MÜNCHEN, München (DE); KARLSRUHER INSTITUT FÜR TECHNOLOGIE, Karlsruhe (DE)

(72) Inventors: Sonja BERENSMEIER, München (DE); Silvia BLANK-SHIM, München (DE); Sebastian SCHWAMINGER, München (DE); Paula FRAGA GARCIA, Freising (DE); Wolfgang WENZEL, Weingarten (DE); Karin FINK, Bruchsal (DE); Priya ANAND, Karlsruhe (DE); Monika BORKOWSKA-PANEK, Wiesloch (DE)

(21) Appl. No.: 16/333,959

(22) PCT Filed: Sep. 15, 2016
(86) PCT No.: PCT/EP2017/073291

§ 371 Date: Mar. 15, 2019

(30) Foreign Application Priority Data
Sep. 16, 2016 (EP) 16189240.1
Sep. 21, 2016 (EP) 16189914.1

(51) Int. Cl. G01N 33/543; C07K 7/06; C07K 7/08

(52) U.S. Cl. CPC G01N 33/54333; G01N 2446/00; C07K 7/08; C07K 7/06

(57) ABSTRACT

The present invention relates to a peptide consisting of a sequence of 5 to 30, preferably 6 to 12, most preferably 10 to 12 amino acids, wherein (a) at least ⅔ of said amino acids have a functional group or side chain which is negatively charged at neutral pH; (b) amino acids which do not have a functional group or side chain which is negatively charged at neutral pH, if present, meet one or both of requirements (i) and (ii): (i) none of them has a functional group or side chain which is positively charged at neutral pH; and (ii) at least one of them has a side chain which does not bear a net charge at neutral pH or which has a functional group or side chain that does not bear a net charge at neutral pH.

The present invention relates to a peptide consisting of a sequence of 5 to 30, preferably 6 to 12, most preferably 10 to 12 amino acids, wherein (a) at least ⅔ of said amino acids have a functional group or side chain which is negatively charged at neutral pH; (b) amino acids which do not have a functional group or side chain which is negatively charged at neutral pH, if present, meet one or both of requirements (i) and (ii): (i) none of them has a functional group or side chain which is positively charged at neutral pH; and (ii) at least one of them has a side chain which does not bear a net charge at neutral pH or which has a functional group or side chain that does not bear a net charge at neutral pH.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Magnetic nanoparticles (MNPs) are widely used for purification of proteins, nucleic acids and other biological molecules (Fraga Garcia, P., et al. (2015): High-gradient magnetic separation for technical scale protein recovery using low cost magnetic nanoparticles, Separation and Purification Technology 150, pp. 29-36; Rittich, B. et al. (2013): SPE and purification of DNA using magnetic particles, Journal of Separation Science 36 (15), pp. 2472-2485; Colombo, M., et al. (2012): Biological applications of magnetic nanoparticles, Chem. Soc. Rev. 41 (11), pp. 4306-4334). MNP are also useful for biomedical applications such as optical imaging (Le Sage, D., et al. (2013): Optical magnetic imaging of living cells, Nature 496 (7446), pp. 486-489), drug delivery and hyperthermia and, as well as contrast agents for magnetic resonance imaging (Boyer, C., et al. (2010): The design and utility of polymer-stabilized iron-oxide nanoparticles for nanomedicine applications, NPG Asia Mater 2, pp. 23-30; Shinkai, M. (2002): Functional magnetic particles for medical application, Journal of Bioscience and Bioengineering 94 (6), pp. 606-613; Senpan, A., et al. (2009): Conquering the Dark Side: Colloidal Iron Oxide Nanoparticles, ACS Nano 3 (12), pp. 3917-3926)). Small magnetite particles naturally occur in magnetotactic bacteria, helping the hosts to orient themselves in the geomagnetic field (Mann, S., et al. (1984): Structure, Morphology and Crystal-Growth of Bacterial Magnetite, Nature 310 (5976), pp. 405-407).

MNP can be manufactured in sizes ranging from a few to hundreds of nanometers, which results in a large specific surface area. MNPs show superparamagnetic behavior, which allows their manipulation by an external magnetic field (Pankhurst, Q. A., et al. (2003): Applications of magnetic nanoparticles in biomedicine, Journal of Physics D: Applied Physics 36 (13), pp. R167; Zhao, F. Y., et al. (2012): Preparation and magnetic properties of magnetite nanoparticles, Mater Lett 68, pp. 112-114).

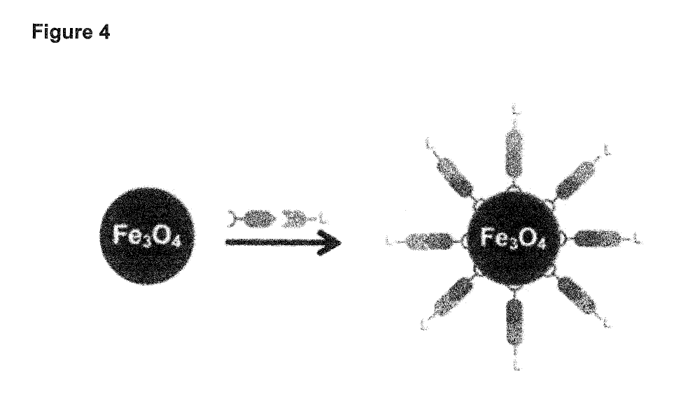

In order to be used in a number of applications, MNPs must be functionalized to selectively bind to theft respective interaction partners. This can presently be achieved by attaching metal ion chelating molecules like nitrilotriacetic acid to the MNP surface so that His-tagged biomolecules can be bound (Block, H. et al. 2009: Chapter 27, Immobilized-Metal Affinity Chromatography (IMAC); Richard R. Burgess, Murray P. Deutscher (Eds.): Guide to protein purification, vol. 463. 2nd ed. Amsterdam, Boston: Elsevier/Academic Press (Methods in Enzymology, v. 463), pp. 439-473). Drawbacks of this method are the leakage of toxic metal ions and instability of the surface functionalization (Gaberc-Porekar, V. and Menart, V. 2005: Potential for Using Histidine Tags in Purification of Proteins at Large Scale; Chem. Eng. Technol. 28 (11), pp. 1306-1314); Hearn, M. and Acosta, D. 2001: Applications of novel affinity cassette methods: use of peptide fusion handles for the purification of recombinant proteins; in J. Mol. Recognit. 14 (6), pp. 323-369). Other surface modifications for protein adsorption on magnetic particles use glutathione, streptavidin, biotin or protein A, but all of these lead to high costs of over 400 to several thousand euros per gram (Franzreb, M. et al. 2006: Protein purification using magnetic adsorbent particles; in Appl Microbiol Biol 70 (5), pp. 505-516). This principle is illustrated in FIG. 4. Problems of covalent immobilization techniques such as amino-silanization are the high complexity (Uzun, K. et al. 2010: Covalent immobilization of invertase on PAMAM-dendrimer modified superparamagnetic iron oxide nanoparticles; in Journal of Nanoparticle Research 12 (8), pp. 3057-3067) and loss of enzymatic activity due to the reaction of catalytic amino acids (Johnson, A. et al. 2008: Novel method for immobilization of enzymes to magnetic nanoparticles; in J Nanopart Res 10 (6), pp. 1009-1025).

For these reasons it would be advantageous to overcome the requirement of application-specific functionalization and to engineer tags such as peptide sequences that can bind to MNPs and, in case of the tags being peptides, can directly be encoded by those nucleic acids which also encode the polypeptide of interest for a given application.

In view of the deficiencies of the prior art, the technical problem underlying the present invention can be seen in the provision of improved means and methods of binding molecules such as biomolecules or analytes in general to iron oxide-containing surfaces, especially magnetic particles. This technical problem is solved by the subject-matter of the attached claims.

Accordingly, in the first aspect, the present invention relates to a peptide consisting of a sequence of 5 to 30, preferably 6 to 12, most preferably 10 to 12 amino acids, wherein (a) at least ⅔ of said amino acids have a functional group or side chain which is negatively charged at neutral pH; (b) amino acids which do not have a functional group or side chain which is negatively charged at neutral pH, if present, meet one or both of requirements (i) and (ii): (i) none of them has a functional group or side chain which is positively charged at neutral pH; and (ii) at least one of them has a side chain which does not bear a net charge at neutral pH or does not bear a net charge at neutral pH.

In particular, and as stated further below, the present invention relates to a peptide consisting of a sequence of 5 to 30, preferably 6 to 12, most preferably 10 to 12 amino acids, wherein (a) at least ⅔ of said amino acids have a functional group or side chain which is negatively charged at neutral pH; (b) amino acids which do not have a functional group or side chain which is negatively charged at neutral pH, if present, meet one or both of requirements (i) and (ii): (i) none of them has a functional group or side chain which is positively charged at neutral pH; and (ii) at least one of them has a side chain which does not bear a net charge at neutral pH or which has a functional group or side chain that does not bear a net charge at neutral pH.

The term “peptide” has its art-established meaning. It relates to a polycondensate of amino acids, the number of amino acids being 30 or less. In accordance with the invention, peptides have length between 5 and 30 amino acids. Envisaged lengths are 5, 6, 7, 8, 9, 10, 11, 12, 13, 14, 15, 16, 17, 18, 19, 20, 21, 22, 23, 24, 25, 26, 27, 28, 29 and 30 amino acids. Peptides may be chemically synthesized or translated from nucleic acids encoding them.

The term “peptide” as used herein does not imply a limitation to α-amino acids. Therefore, the charge status of the amino acids is a feature of a functional group comprised in a given amino acid. To the extent the amino acids are α-amino acids, the term “side chain” has its usual meaning and designates group R in the following generic formula: —NH—CHR—CO—. The side chain may carry a functional group which is charged at neutral pH.

Amino acids are preferably α-amino acids, wherein the 20 proteinogenic amino acids are especially preferred. To the extent use is made of proteinogenic amino acids, peptides of the present invention as well as fusion constructs, namely polypeptides and proteins as disclosed further below can be encoded by nucleic acids. Having said that, it is also envisaged to use other amino acids, wherein said other amino acids include α-amino acids which do not belong to the set of 20 proteinogenic amino acids such as pyrrolysine and selenocysteine. Among α-amino acids, preference is given to L-amino acids. At the same time, the use of D-amino acids is also envisaged. Moreover, one or more amino acids which are not α-amino acids, such as β-amino acids may be present. Preference is given to absence of non-proteinogenic amino acids. Yet, any of the above-mentioned non-proteinogenic amino acids may be present, preferably in small numbers such as 1, 2, 3, 4 or 5. It is not excluded that also 6, 7, 8, 9 or 10 non-proteinogenic amino acids are comprised in peptides in accordance with the first aspect of the invention. Peptides of the invention may entirely consist of non-proteinogenic amino acids.

In accordance with the first aspect, at least ⅔ of the amino acids are required to be amino acids with a functional group or side chain that is negatively charged at neutral pH. It is understood that at least in a preferred embodiment said amino acids do not contain a functional group or side chain which is positively charged at neutral pH.

Among the proteinogenic amino acids, this requirement is fulfilled by Asp and Glu. If a peptide consists of 9 amino acids, this means that, in accordance with requirement (a), 6, 7, 8 or all 9 amino acids have a side chain which is negatively charged at neutral pH.

To the extent amino acids are comprised in peptides of the invention which do not fulfill requirement (a), item (b) of the first aspect imposes restrictions on these further amino acids. It follows from conditions (i) and (ii) that up to ⅓ neutral or polar amino acids are possible. If both requirements (i) and (ii) are fulfilled, no amino acids are present which are positively charged at neutral pH. To the extent one or more amino acids with a side chain that is positively charged at neutral pH are present, this entails the requirement for a presence of at least one amino acid which does not bear a net charge at neutral pH or which has a functional group or side chain that does not bear a net charge at neutral pH. If the sequence of a peptide in accordance with the first aspect does not contain any amino acid with a side chain that is positively charged at neutral pH, there are no further limitations on its composition other than the limitation of (a).

It is understood that at least in a preferred embodiment said amino acids which have a functional group or side chain that is positively charged at neutral pH do not contain a functional group or side chain which is negatively charged at neutral pH.

Preferred amino acids which are positively charged at neutral pH are Lys, Arg and His.

Whether or not a given amino acid meets any one of the above criteria, may determined by assessing an aqueous solution of the amino acid at issue in its monomeric form, i.e., with free amino and carboxy terminus. Alternatively, N-terminus and C-terminus may be protected, for example, the N-terminus may be acetylated and the C-terminus may amidated. pKₐ value(s) of an amino acid can be determined by acid/base titration. The pKₐ value in turn determines the charge status at a given pH value.

Preferred specific amino acids meeting the above requirements are the subject of preferred embodiments which are disclosed further below.

Related to the first aspect, the present invention also provides, in a second aspect, a peptide consisting of a sequence of 5 to 30, preferably 6 to 12, most preferably 10 to 12 amino acids, wherein said sequence consists of at least one (S) region and, optionally, at least one (W) region, wherein, if at least one (W) region is present, the (S) and (W) regions are alternating; with (S) representing a region consisting of negatively charged and/or positively charged amino acids, and (W) representing a region consisting of neutral, hydrophobic and/or polar amino acids, wherein (a) each (S) region independently consists of more than 2 negatively charged and/or more than 2 positively charged amino acids, (W) regions independently consist of 1, 2 or 3 amino acids; and the total number of positively charged and negatively charged amino acids in said sequence is greater than 5; or (b) each (S) region independently consists of more than 2 negatively charged and/or 0, 1 or 2 positively charged amino acids; (W) regions independently consist of 1, 2 or 3 amino acids; the total number of positively charged amino acids in said sequence is 0, 1 or 2; the total number of negatively charged amino acids in said sequence is at least 5; and said peptide has an isoelectric point (pI) of less than about 5.0.

This aspect of the invention introduces the notion of regions within the sequence of the peptide. In particular, the designations (S) and (W) refer to groups of adjacent amino acids within the peptide of the invention which confer strong binding to a surface comprising iron oxide and groups which consist of adjacent amino acids which confer weak binding to such surface, respectively.

Designations of amino acids relating to the charge status, especially the designations “negatively charged”, “positively charged” and “neutral” refer to the charge status at neutral pH. The same applies to polar amino acids.

The distinction between items (a) and (b) is inter alia based on the number of allowed amino acids bearing a positive charge. Item (a) allows for more than two positively charged amino acids per (S) region, whereas item (b) confines the number of positively charged amino acids per (S) region to 2. This will be discussed in more detail further below. Higher numbers of positively charged amino acids in accordance with item (a) confer particularly strong binding to surfaces comprising iron oxide. Such particularly strong binding can be viewed, for practical purposes, as being irreversible. This is also illustrated in the Examples enclosed herewith.

On the other hand, by confining the number of positively charged amino acids to lower values (as in item (b)) provides for reversible binding to the mentioned surfaces. Also the notion of reversibility will be discussed in more detail below.

Examples of alternating (S) and (W) regions include (S)(W), (W)(S), (S)(W)(S), (W)(S)(W), (S)(W)(S)(W) and (W)(S)(W)(S). A preferred pattern is (S)(W)(S). This applies to all aspects of the invention.

In a preferred embodiment of the peptide in accordance with the first aspect, homopolymers of amino acids which have a functional group or side chain which is negatively charged at neutral pH are excluded. Particularly preferred is that homopolymers of Asp and/or of Glu are excluded. Especially preferred is that the homodecamer DDDDDDDDDD is excluded.

In a further preferred embodiment, the fraction of amino acids which have a functional group or side chain which is positively charged at neutral pH is (a) in the range between zero and 0.2, preferably zero; or (b) greater than 0.2.

This preferred embodiment is related to the distinction between items (a) and (b) of the second aspect disclosed above. In particular, a low or zero fraction of amino acids which have a side chain which is positively charged at neutral pH defines reversible binders. Item (b) on the other hand defines irreversible binders. In either case, binding is preferably non-covalent.

An example of a peptide sequence which has a fraction of 0.2 in accordance with item (a) is a sequence consisting of 10 amino acids, 2 of which have a side chain which is positively charged at neutral pH.

In a further preferred embodiment, said at least one amino acid having a side chain which does not bear a net charge at neutral pH, which is an option in accordance with the first aspect, is present and is flanked on either side by at least one, preferably at least two amino acids having a side chain which is negatively charged at neutral pH.

Noting that amino acids with side chains which do not bear a net charge at neutral pH contribute weakly to binding of said peptide to a surface comprising iron oxide, this preferred embodiment defines an implementation of the above-mentioned (S)(W)(S) pattern.

In a further preferred embodiment, said peptide is capable of binding to a surface comprising iron oxide, preferably a magnetic nanoparticle.

The peptides in accordance with the first and the second aspect of the invention render the art-established application-specific functionalization of magnetic nanoparticles dispensable. The art-established functionalization of the surface of nanoparticles does not only entail the need for a specific modification of the nanoparticles for any given specific application, it also is sometimes instable or leaks. In other words, the paradigm depicted in FIG. 4 does not apply to applications employing nanoparticles which make use of peptides of the present invention. This saves time and costs. When performing methods in accordance with the present invention (disclosed further below), magnetic particles can be conveniently recycled many times.

Said surface may have any geometry. It extends to planar and curved surfaces. Preferred are particles such as spherical particles including nanoparticles and spherical nanoparticles. The diameter of such particles may be in the range between 0.1 and 100 nm, preferably between 1 and 50 nm or between 1 and 20 nm including between 1 and 10 nm. Preferred particles are colloidal particles.

Preference is given to magnetic surfaces, especially magnetic nanoparticles. Magnetic nanoparticles are commercially available, e.g. from Sigma Aldrich.

The term “iron oxide” includes any form of iron (Fe) oxide such as FeO, Fe₂O₃ and Fe₃O₄. Iron oxides may also be hydrated. Preferred are magnetic iron oxides, especially Fe₃O₄.

The mentioned surface as well as the mentioned particles may consist of iron oxide or may comprise, in addition to iron oxide, other material such as oxides of other metals. Surfaces comprising mixtures of metal oxides and having magnetic properties are preferred. Preferred other metals are Co, Ni, Mn and Cu which other metals can also be comprised in small quantities only (dotation). Typical other materials are ferrites which have the following structure: XFe₂O₄ where X can be Co, Ni, Mn, Zn, Cu, Mg, Sr, Ba or mixtures of these elements.

In a preferred embodiment, magnetic nanoparticles are prepared in accordance with the methods described in the Examples enclosed herewith.

In a preferred embodiment, binding to said surface, preferably to magnetic nanoparticles, occurs in (a) 50 mM Tris buffer at pH 7.4, 137 mM NaCl and 2.7 mM KCl; (b) in 50 mM MES buffer pH 6, 137 mM NaCl and 2.7 mM KCl; (c) an unbuffered solution at a pH in the range from about 5 to about 7; or (d) acetated buffered saline. The above are preferred binding conditions.

Acetate buffered saline comprises 50 mM sodium acetate buffer at pH 7, 137 mM NaCl and 2.7 mM KCl.

In a preferred embodiment of the above-defined reversible binders, said binding is reversible in 50 mM citrate, 137 mM NaCl and 2.7 mM KCl at pH=6. These are preferred dissociation conditions. Preferably, reversible binders in accordance with the present invention are characterized in that under the above-disclosed conditions at least ⅔ of bound peptide molecules are set free.

In either case, i.e. in relation to both binding conditions and dissociation conditions, it is preferred, but not required, that further agents are present. These further agents include surfactants and stabilizers. Preferred surfactants are non-ionic surfactants. Preferred non-ionic surfactants are sorbitan fatty acid esters including polyoxyethylenesorbitan fatty acid esters, such as Tween including Tween20.

In a further preferred embodiment, (a) one, more or all of said amino acids which have a side chain which is negatively charged at neutral pH are selected from Asp and Glu; and/or (b) one, more or all amino acids, to the extent present, which have a side chain which does not bear a net charge at neutral pH are selected from Gly, Ser, Ala, Asn, Cys, Gln, His, Ile, Leu, Met, Phe, Pro, Thr, Trp, Tyr, Val and selenocystein, Gly being preferred.

Especially preferred peptides in accordance with the present invention include the following:

(SEQ ID NO: 1)
EEEEEE
(SEQ ID NO: 2)
DDDDDD
(SEQ ID NO: 3)
DDDDEDDEDDDD
(SEQ ID NO: 4)
DDDEDDEDDEDD
(SEQ ID NO: 5)
DDDDDDDDDD
(SEQ ID NO: 6)
DDDDDEDDDDDD
(SEQ ID NO: 7)
DDDEDDEDDD
(SEQ ID NO: 8)
DDDDDDDDDDDD
(SEQ ID NO: 9)
DDEDDEDDED
(SEQ ID NO: 10)
DDDDEDDDDD
(SEQ ID NO: 11)
DDDDDGGDDDDD
(SEQ ID NO: 12)
DDDDDDDD
(SEQ ID NO: 13)
DDDDGGDDDD
(SEQ ID NO: 14)
DDDDGGGGDDDD
(SEQ ID NO: 15)
DDDGGGGDDD
(SEQ ID NO: 16)
DDDGGDDD
(SEQ ID NO: 17)
DDDGGGGGGDDD
(SEQ ID NO: 18)
DDDDDDDDDDD
(SEQ ID NO: 19)
EEEEGGGGEEEE

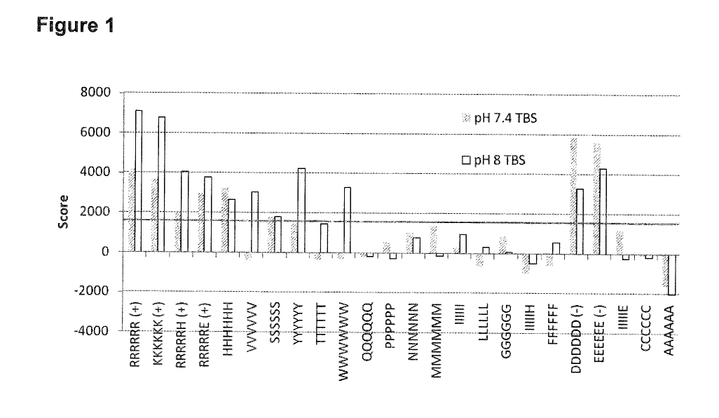

Inter alia, the sequences of SEQ ID NOs: 1 and 2 have been experimentally characterized; see Example 2 as well as FIG. 1.

SEQ ID NO: 11 is an example of a sequence having a (S)(W)(S) pattern, wherein the first (S) region is DDDDD, the (W) region is GG, and the second (S) region is DDDDD. Further preferred specific sequences in accordance with the present invention which follow the (S)(W)(S) pattern are SEQ ID NOs: 13 to 17.

In a preferred embodiment, increasing the length of the (W) region in a sequence having an (S)(W)(S) pattern, especially by increasing the number of Gs, while maintaining the overall number of amino acids in the two (S) regions constant, said amino acids in said (S) regions preferably being exclusively D or exclusively E, is a means of enhancing binding to surfaces containing iron oxide.

Further preferred peptides are homooligomers of D or E. Envisaged numbers of monomers in said homooligomers are 5, 6, 7, 8, 9, 10, 11, 12, 13, 14, 15, 16, 17, 18, 19, 20, 21, 22, 23, 24, 25, 26, 27, 28, 29 and 30.

In a third aspect, the present invention provides a molecule which is a polypeptide or protein, said polypeptide or protein comprising at least one sequence of a peptide as defined in accordance with the first or second aspect of the present invention, wherein the sequence(s) in said polypeptide or protein which do(es) not comprise said at least one sequence of a peptide are heterologous with respect to said sequence of a peptide.

It is understood that the above language “polypeptide or protein comprising at least one sequence of a peptide” relates to fusion constructs. Peptides in accordance with the present invention may be fused N-terminally and/or C-terminally to a polypeptide or protein. Also, it is envisaged to insert the sequence of a peptide in accordance with the invention into a polypeptide or protein such that on either side of the peptide of the invention there are flanking sequences, said flanking sequences originating from said polypeptide or protein. As mentioned above, the advantage of fusion constructs is that they can be obtained by transcription and translation of nucleic acids. For such molecule to be encoded by a nucleic acid, it is understood that polypeptide or protein as well as peptide consist exclusively of amino acids which can be encoded by a nucleic acid. Typically, these are the 20 proteinogenic amino acids.

Accordingly, the present invention also provides a nucleic acid encoding a peptide of the first or second aspect or a molecule in accordance with the third aspect of the invention. The nucleic acid may consist of a nucleotide sequence encoding said peptide or said molecule or may comprise such coding sequence and furthermore comprise flanking sequences. A preferred implementation of a nucleic acid comprising also flanking sequences is an expression vector such as a pET vector. In addition to a vector which contains a nucleic acid sequence encoding a molecule or fusion construct of the invention, also expression vectors are provided which contain one or more nucleic acid sequences encoding a peptide in accordance with the first or second aspect of the invention and a cleavage site for a restriction enzyme. Such cleavage site permits the insertion of a nucleic acid encoding a polypeptide or protein of interest.

It is apparent from the definition of the molecule in accordance with the third aspect that naturally occurring polypeptides and proteins do not fall under the term “molecule” in accordance with the present invention. Instead, there is the requirement that the peptide on the one hand and the above-mentioned flanking sequences on the other hand are heterologous to each other. Similarly, the sequence of said polypeptide or protein as a whole is required to be heterologous with respect to the sequence of the peptide under consideration in accordance with the first or second aspect.

It is furthermore envisaged to use linker sequences which connect the peptide in accordance with the first or second aspect of the invention to a polypeptide or protein or, to the extent the sequence of said peptide is inserted into said polypeptide or protein, to a partial sequence of said polypeptide or protein. Preferably, such linker sequences, to the extent they are present, are heterologous with regard to said polypeptide or protein and furthermore heterologous with regard to the sequence of said peptide in accordance with the first or second aspect. Preferred linker sequences include oligoglycine sequences and the sequence SSG.

In a fourth aspect, the present invention provides a molecule covalently bound to at least one sequence of a peptide in accordance with the first aspect (or in accordance with the second aspect) of the present invention, wherein the covalent bond is not a main chain peptide bond, wherein said molecule is preferably a polypeptide or protein or a nucleic acid such as DNA or RNA including siRNAs. Further envisaged molecules are organic molecules including small organic molecules with a molecular weight between 100 and 1000 Da and/or pharmaceuticals which are organic molecules; ligands including ligands of naturally occurring receptors; and toxins or toxoids.

This aspect relates to conjugates. Conjugates are herein distinguished from fusion constructs. Accordingly, the moiety which is conjugated to a peptide in accordance with the present invention may be a polypeptide or protein, but does not have to. The moiety to be conjugated may be conjugated to the N-terminus of the peptide of the invention. Ethyl-dimethylaminopropyl-carbodiimid (EDC) coupling may be used.

In preferred embodiments of the molecule in accordance with the third and the fourth aspect, said polypeptide or protein is (a) an antibody or fragment thereof; or (b) an enzyme or hormone.

In a fifth aspect, the present invention provides a surface comprising iron oxide, preferably a magnetic nanoparticle, which is bound to at least one peptide or molecule as defined in accordance with the first, second, third or fourth aspect.

The present invention also relates to uses of the surface in accordance with the fifth aspect, said uses being for a biotechnological process. In a preferred embodiment of said use, said molecule is an enzyme and said biotechnological process is or comprises a reaction catalyzed by said enzyme.

Related thereto, the present invention also provides a method for enzymatic processing of a substrate, said method comprising: (a) bringing into a contact a surface in accordance with the fifth aspect, wherein said molecule is an enzyme, with a substrate of said enzyme. Preferably, said contacting is effected under conditions that permit conversion of said substrate into a product, the conversion being catalyzed by said enzyme. Such conditions are generally known in the art and may be specific for a given enzyme/substrate pair.

In a preferred embodiment of said method, said method further comprises one or more of the following: (b) separating said surface from the reaction mixture; (c) enriching or purifying the obtained product.

In a sixth aspect, the present invention provides a method of separating a molecule as defined in accordance with the third or fourth aspect from a sample, said method comprising: (a) contacting said sample with a surface comprising iron oxide, preferably magnetic nanoparticles, under conditions allowing binding of said molecule to said surface; and (b) separating said surface from said sample; thereby separating said molecule from said sample.

Given that the present invention provides peptides as well as molecules which are capable of binding to a surface-containing iron oxide, the skilled person can determine without further ado the conditions in accordance with item (a) of this aspect. Preferred conditions are disclosed further below.

Separating in accordance with step (b) of the method of the sixth aspect can be done by any means. To the extent magnetic surfaces, magnetic particles and magnetic nanoparticles are used, said separating is preferably effected with a magnet.

Devices for separating magnetic particles from mixtures are available from various manufacturers including Thermo Fisher.

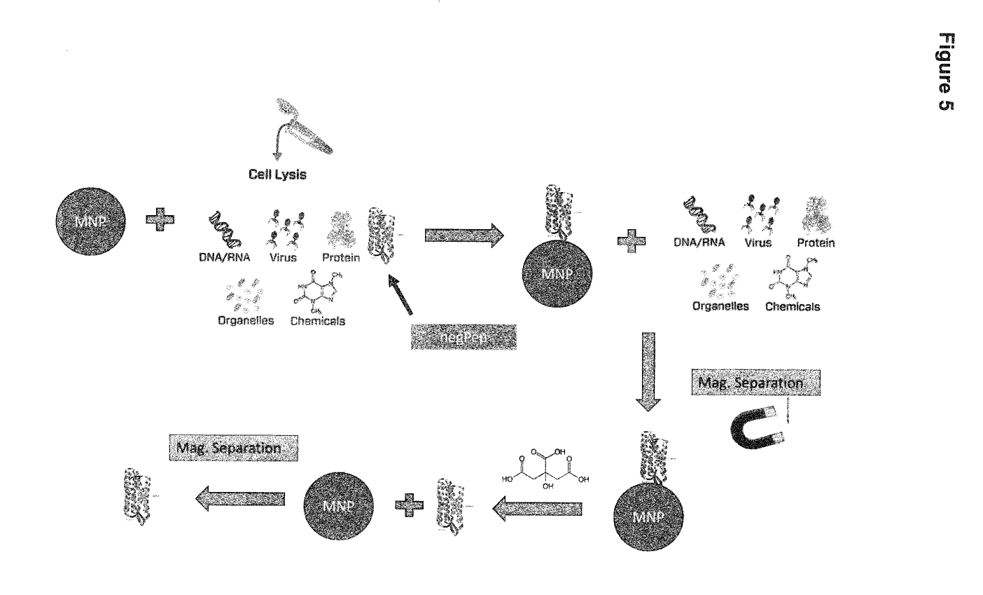

FIG. 5 illustrates an exemplary manner the method in accordance with the sixth aspect.

In a seventh aspect, the present invention provides a method of separating an analyte from a sample, said analyte being capable of binding to a molecule in accordance with the third or fourth aspect, said method comprising: (a) bringing into contact said sample, said molecule and a surface comprising iron oxide, preferably magnetic nanoparticles, under conditions allowing (i) binding of said molecule to said surface and (ii) binding of said analyte to said molecule; and (b) separating said surface from said sample; thereby separating said analyte from said sample.

As noted above, the skilled person, provided with the information of the present disclosure, can determine without further ado the conditions in accordance with item (i) of step (a) of the method of the seventh aspect.

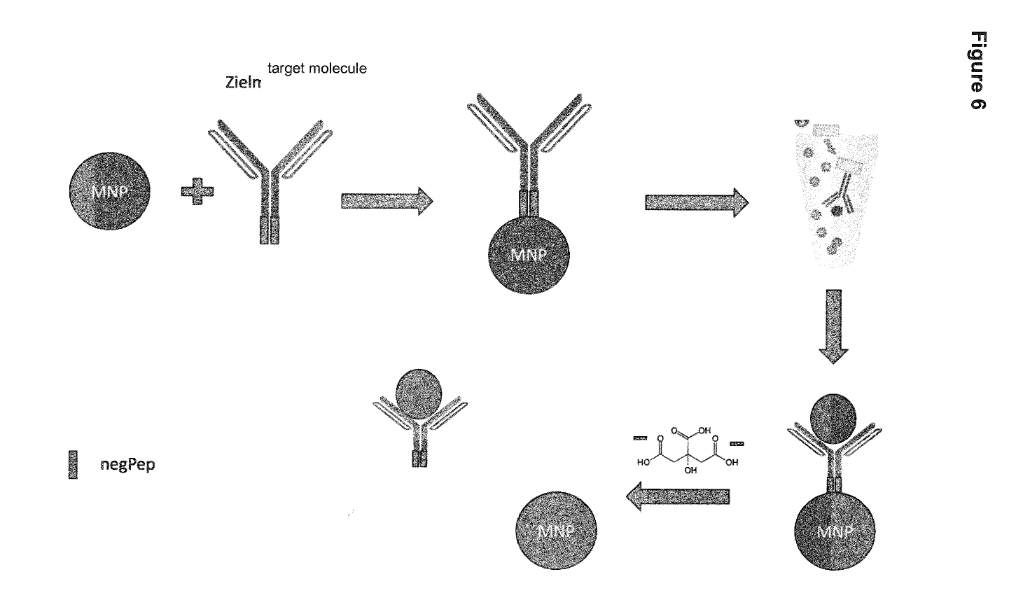

Conditions in accordance with item (ii) are generally known in the art. To give an example, if said analyte is an antigen or a molecule comprising an epitope recognized by a given antibody, and said molecule is an antibody, the antibody in turn comprising a peptide in accordance with the first or second aspect of the present invention, the known conditions which allow binding of the epitope to the antibody define the conditions in accordance with item (ii). Fine-tuning and/or optimizing such conditions can be done by the skilled person without further ado, for example by slightly varying conditions and determining the performance upon such variation. The method in accordance with the seventh aspect is illustrated in FIG. 6.

In a preferred embodiment, step (a) is implemented by (a)(i) bringing into contact said molecule and a surface comprising iron oxide, preferably magnetic nanoparticles, followed by (a)(ii) bringing into contact the result of (a)(i) with said sample.

In a preferred embodiment of the methods of the present invention, that methods further comprise (c) washing said surface; and/or (d)(i) dissociating said molecule from said surface; (ii) optionally followed by removing said surface from the result of (i).

In a preferred embodiment, said dissociating in step (d)(i) is performed by adding (a) a carboxylic acid, preferably a carboxylic acid comprising two or three carboxylic groups, most preferably citric acid, said carboxylic acid preferably being in buffered solution, most preferably under the following conditions: 50 mM citrate, 137 mM NaCl and 2.7 mM KCl at pH=6; and/or (b) an inorganic oxo acid, preferably phosphoric acid, said oxo acid preferably being in buffered solution.

A further preferred carboxylic acid is tartaric acid. A further preferred carboxylic acid is oxalic acid.

In a further preferred embodiment of the method in accordance with the seventh aspect, the method further comprises (e) dissociating said analyte from said molecule.

Since the interaction between analyte and molecule will generally be known, also conditions suitable for said dissociating are known to the skilled person or can be determined without further ado.

Related to the seventh aspect, the present invention also provides, in an eighth aspect, a method of determining presence or absence of an analyte in a sample suspected of comprising said analyte, said method comprising steps (a) and (b) as defined in relation to a seventh aspect, optionally one, more or all of steps (c), (d)(i), (d)(ii) and (e) as defined in preferred embodiments of the present invention herein above, thereby obtaining a mixture, and (f) analyzing said mixture for the presence of said analyte.

Said analyzing can be performed with any known analytic method. Preferred methods include mass spectrometry and liquid chromatography such as HPLC.

In a preferred embodiment of the methods in accordance with the sixth, seventh and eighth aspect of the present invention, said conditions in step (a) are (a) Tris buffer at a pH between about 7 and about 8; (b) MES buffer at a pH between about 5.8 and about 6.5; or (c) an unbuffered solution at a pH in the range from about 5 to about 7; or (d) acetate buffered saline and preferably as defined in relation to binding of peptides of the first aspect to a surface comprising iron oxide. Any of said conditions preferably include a temperature in the interval between 18 and 35° C., more preferably 20° C. or room temperature.

Further suitable conditions include HEPES buffer, HEPPS buffer, MOPS buffer, TES buffer, TAPS buffer and PIPES buffer, each of them buffers having a pH within plus or minus 1 of the pKₐ value of the respective buffer substance.

In a ninth aspect, the present invention provides a kit comprising or consisting of (a)(i) a peptide in accordance with the first aspect; (ii) a molecule in accordance with the third or fourth aspect of the present invention; and/or (iii) a nucleic acid encoding a peptide of (i) or a molecule of (ii), wherein said molecule is a polypeptide or protein, wherein said nucleic acid is preferably comprised in an expression vector.

In a preferred embodiment, the kit of the present invention comprises one or more or all of the following (b) a surface comprising iron oxide, preferably magnetic nanoparticles; (c) a first solution or constituents for preparing said first solution, said first solution being capable of establishing conditions which allow binding of said peptide and/or said molecule to said surface; (d) a second solution or constituents for preparing said second solution, said second solution being capable of establishing conditions which allow dissociating said peptide and/or said molecule from said surface; and (e) instructions for use of said kit, preferably for performing a method of the present invention.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The Figures show:

FIG. 1: Binding scores of magnetic nanoparticles on peptides at pH 7.4 and pH 8 in Tris buffered saline with 0.25% Tween 20 (T-TBS). The particle suspensions were disaggregated in an ultrasound bath for 15 min before incubation with the membrane. The results are the averages of two membranes tested in the same experiment. The horizontal line indicates the noise level in this experiment.

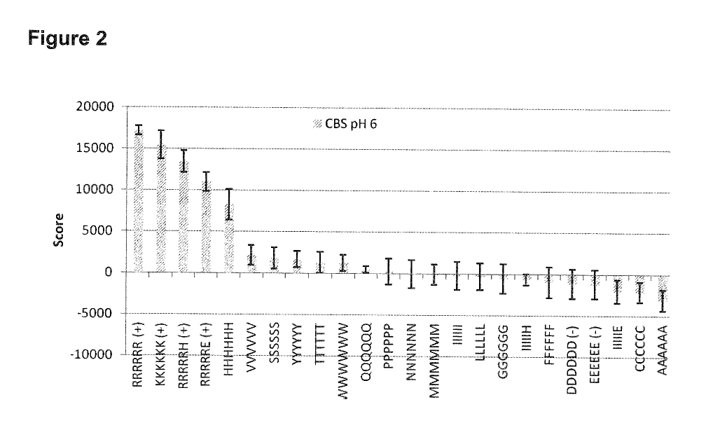

FIG. 2: Binding affinities of magnetic nanoparticles to peptides in Citrate buffered saline pH 6 with 0.25% Tween 20 (T-CBS). The particle suspensions were disaggregated in an ultrasound bath for 15 min before incubation with the membrane. The horizontal line stands for the background noise level.

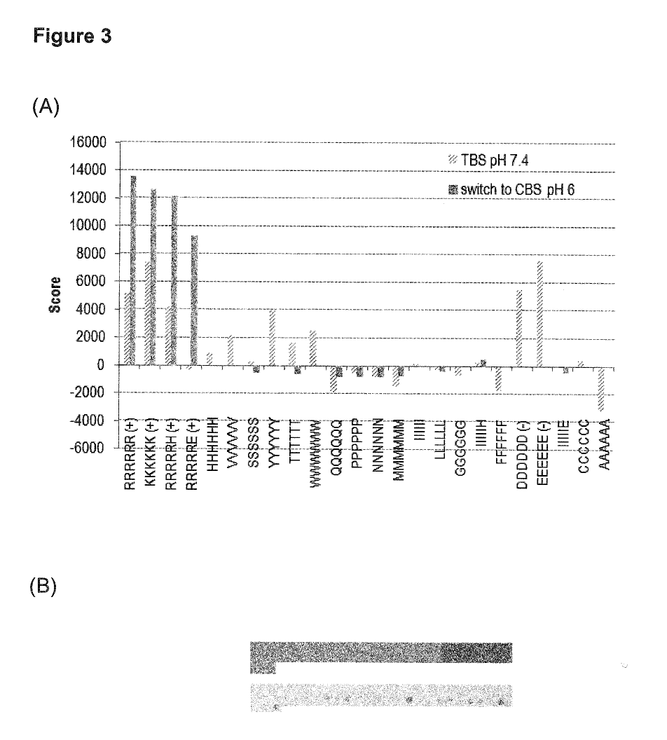

FIG. 3: Binding scores of MNPs on peptide spots after incubation in T-TBS pH 7.4 for 1 h and after transfer of membrane to T-CBS pH 6. (A) The noise level is represented by the horizontal line. (B) Image of a membrane with spots that bound to MNPs being marked.

FIG. 4: In accordance with art-established methods, prior to any application, magnetic nanoparticles have to be functionalized in a tailored manner. In the figure, functionalization of the nanoparticle requires coating thereof with the molecules displayed in dark grey.

FIG. 5: Purification of polypeptides or proteins tagged with peptides of the present invention with magnetic nanoparticles. Peptides of the present invention are fused to the polypeptide or protein of interest and MNPs are added to the initial mixture. MNPs bind to the polypeptide or protein and can be separated by using a magnet. Upon changing the conditions, the polypeptide or protein can be separated from the magnetic nanoparticles. The magnetic nanoparticles in turn can be separated and re-used again.

FIG. 6: An antibody having peptides in accordance with the present invention fused to its F_{c} portion binds to magnetic nanoparticles. Antibody-loaded nanoparticles can be used to separate molecules comprising the cognate epitope from a mixture. When peptides in accordance with the present invention are used which bind reversible to magnetic nanoparticles, the antibody-antigen complex can be detached from the magnetic nanoparticle by changing the conditions. As in the preceding figures, magnetic nanoparticles can be re-used.

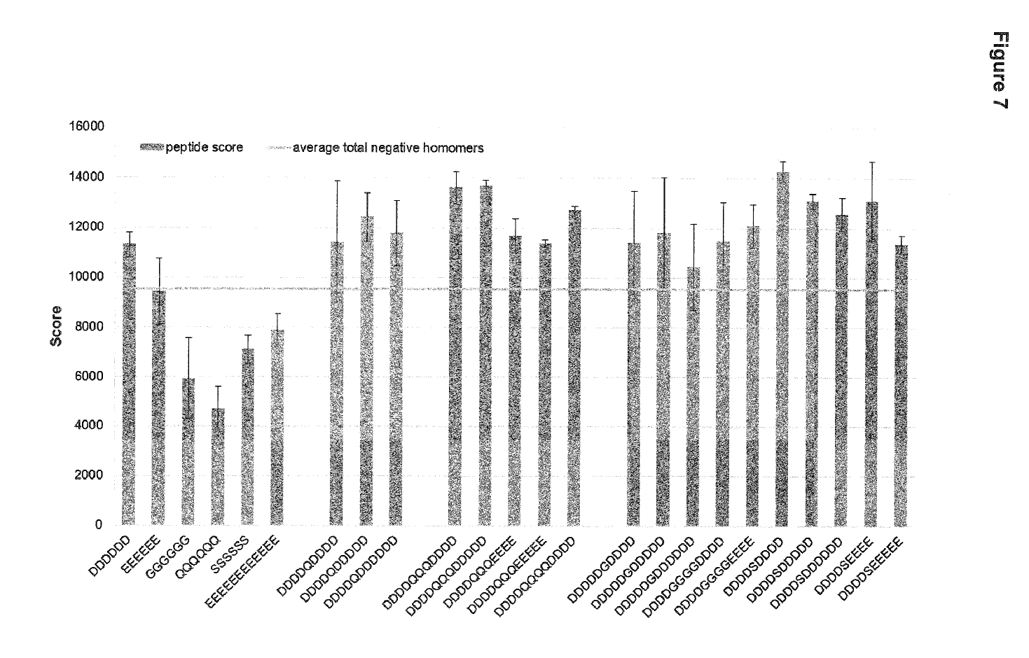

FIG. 7: Interaction data for peptides on a PEPperCHIP®-Array with magnetic nanoparticles in ddH2O at pH 4. Data are averages for duplicates of the same experiment.

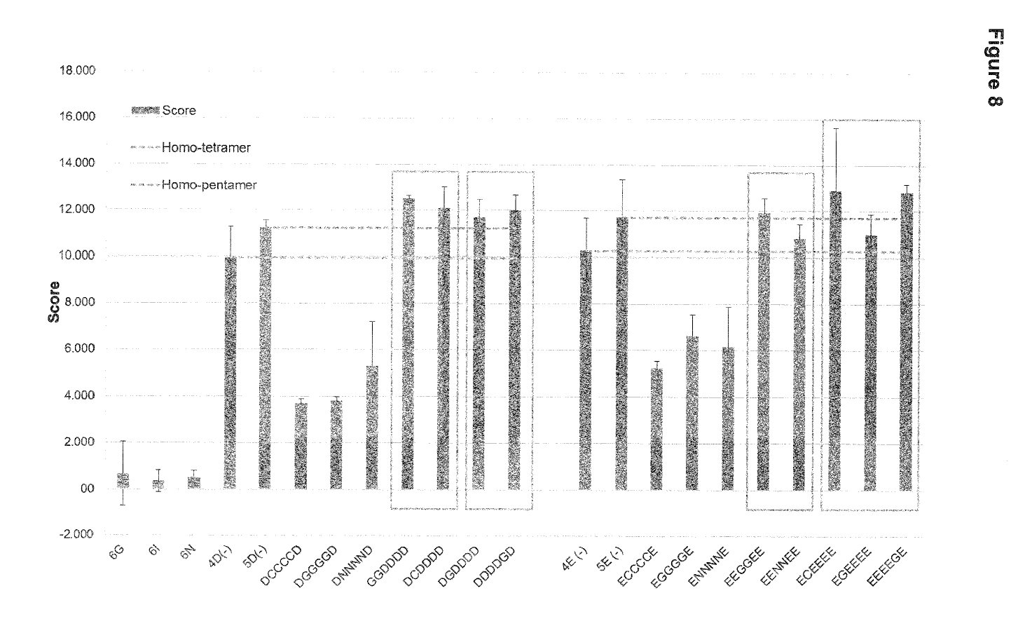

FIG. 8: Scores for the binding of peptides to magnetic nanoparticles in Tris buffered saline (T-TBS) at pH 7.4 on cellulose membranes from Intavis. The data represent averages of two experiments.

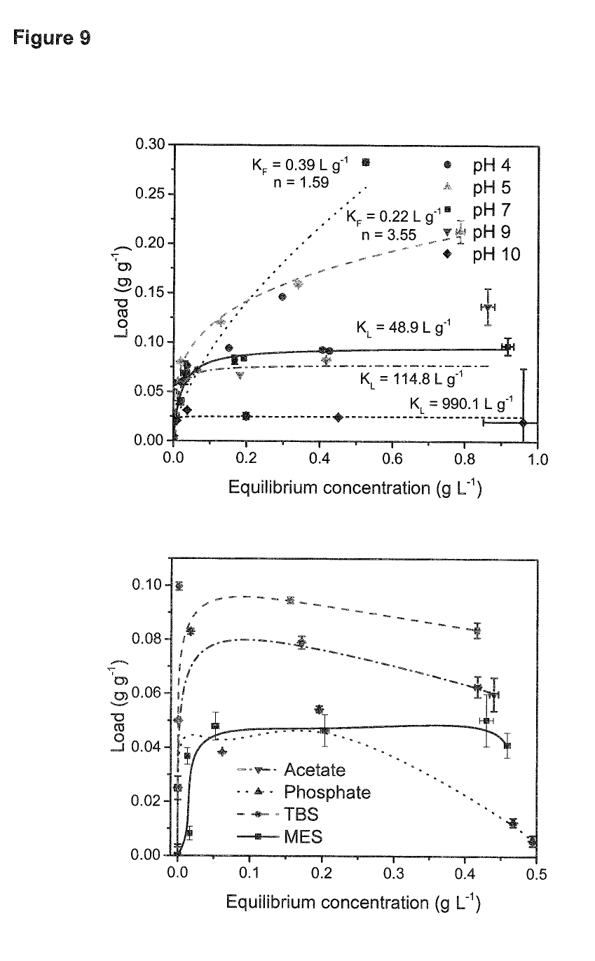

FIG. 9: Adsorption isotherms of octa-glutamic acid on magnetite nanoparticles (1 g L⁻¹) at different pH (top) and in different buffers at pH 7 (bottom), all without Tween. Adsorption isotherms at pH 4.5 and 7 are fitted with a Langmuir function, while the isotherms at pH 9 and 10 are fitted with a Freundlich function.

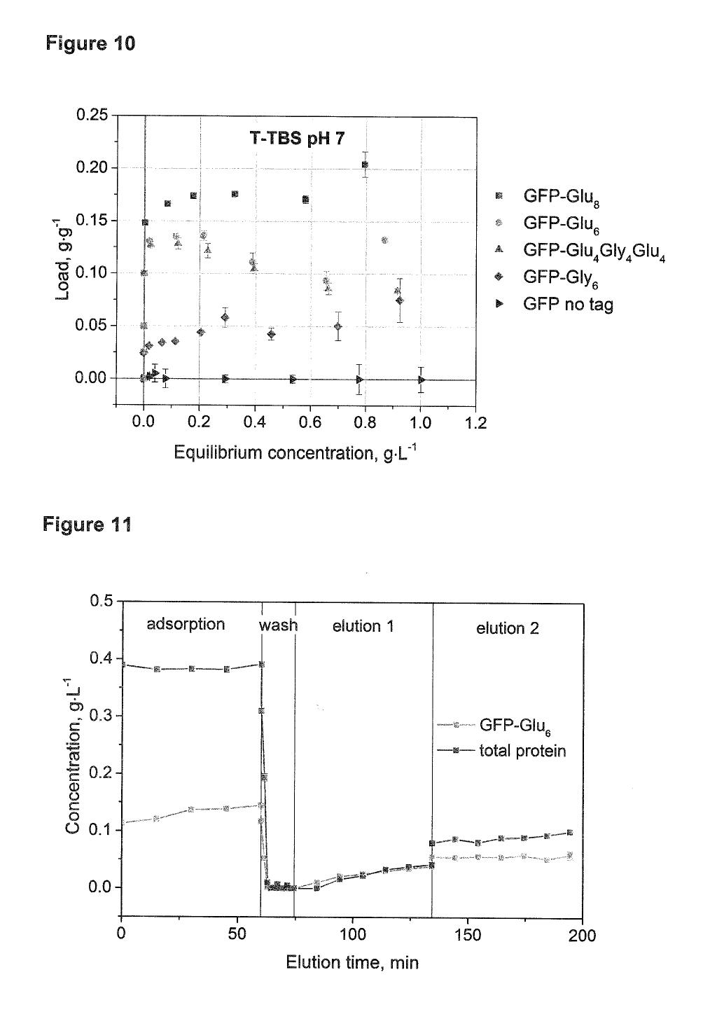

FIG. 10: Adsorption isotherms of tagged green fluorescent protein (GFP) on magnetic nanoparticles in Tris buffered saline (TBS) at pH 7 with 0.1% Tween (T-TBS).

FIG. 11: Magnetic separation of Glu₆-tagged protein from bacterial lysate in TBS at pH 7 using a high gradient magnetic separator. Elution has been effected with citrate buffered saline (CBS) at pH 7.

The Examples illustrate the invention.

### EXAMPLE 1

### Materials and Methods

### Synthesis of Magnetic Nanoparticles

The iron oxide nanoparticles used for this study were synthesized by co-precipitation of Fe²⁺ and Fe³⁺ in alkaline aqueous solutions adapted to the procedure described by Roth et al. (Roth, H.-C., et al. (2015): Influencing factors in the CO-precipitation process of superparamagnetic iron oxide nano particles: A model based study, Journal of Magnetism and Magnetic Materials 377, pp. 81-89). Briefly, 21.2 g of FeCl₃×6 H₂O and 8.29 g of FeCl₂×4 H₂O were dissolved in 200 mL of deionized, degassed water which equals a Fe(III):Fe(II) ratio of 1.88:1. This iron chloride solution was added to 1 L of a 1 M solution of NaOH prepared with deionized, degassed water stirring at 250 rpm in a reaction vessel. The reaction mixture was kept under nitrogen atmosphere at 25° C. and stirred for 30 more minutes before the nanoparticles that had formed were washed several times with deionized water until the conductivity of the MNP solution was below 200 μS cm⁻¹. In order to separate the particles from the washing water the mixture was placed on a NdFeB permanent magnet. FeCl₃×6 H₂O and sodium hydroxide were purchased from AppliChem GmbH, Germany in the highest purity available. FeCl₂×4 H₂O extra pure was obtained from Merck KGaA, Germany.

### Zetapotential Measurements

For zetapotential measurements, a suspension of 0.4 g/L magnetic nanoparticles in buffer was sonicated for 15 minutes. In a Beckman Coulter Delsa Nano C, the zetapotential was determined at 25° C. three times with 10 accumulation times at 5 different positions in a flow cell each at 60 V with a pinhole of 50 μm.

### Magnetic Nanoparticle Binding Assay

In order to determine the binding between peptides and magnetic nanoparticles (MNPs) CelluSpot peptide arrays from Intavis with 5 to 10 nmol of peptides per spot were used. The cellulose membrane on which the peptides had been synthesized by the manufacturer was conditioned with 1 mL of methanol in order to rehydrate hydrophobic peptides (Golemis, E. and Adams, P. D. (2005): Protein-protein interactions. A molecular cloning manual. 2nd ed. Cold Spring Harbor, N.Y.: Cold Spring Harbor Laboratory Press). The buffers employed were T-CBS, T-PBS or T-TBS which is 50 mM citrate, phosphate or tris, respectively, with 0.25% Tween 20, 137 mM NaCl and 2.7 mM KCl. The orbital shaker used for incubation was MulitBio3D from Biosan.

The array was washed three times for 10 min each with 50 mL of buffer. After washing, the membrane was incubated for 60 minutes in a MNP solution (Kuboyama, M., et al. (2012): Screening for silver nanoparticle-binding peptides by using a peptide array, Biochemical Engineering Journal 66 (0), pp. 73-77). The spots on which magnetite bound became dark grey. In order to remove unbound particles the membrane was washed three times for 10 minutes each with buffer (Golemis, E. and Adams, P. D. (2005): Protein-protein interactions. A molecular cloning manual. 2nd ed. Cold Spring Harbor, N.Y.: Cold Spring Harbor Laboratory Press). Then the cellulose membrane was dried overnight at 4° C. and an image of it was taken using a GelDoku station.

### Peptidarray-Experimente

The PEPperCHIP®-Array with printed peptides has been purchased from PEPperPRINT Germany. 0.4 g/L MNP suspended in ddH2O pH 4 have been desagglomerated by ultrasonication for 4 minutes with a Branson sonifier 450D prior to a 1:10 dilution with ddH2O pH 4. This suspension was incubated on the chip with an orbital shaker for 1 h and then dipped into 1 mM Tris buffer pH 7.4. The chip was scanned with a Typhoon FLA 9500 scanner in digitalization mode with a resolution of 10 μm and analyzed with the software PepSlideAnalyzer.

Adsorption Isotherms with Peptides

The octa-glutamic acid peptide (101 mg) was obtained from metabion international AG (Germany) as a lyophilized powder with a purity of >95% and stored at +4° C. prior to use. For the binding experiments different amounts of peptides were incubated with magnetite nanoparticles (1 g L⁻¹) at different pH and buffer conditions for at least one hour at 25° C. under vigorous shaking. The supernatant was separated from the particles with hand magnets, decanted and analysed with an Infinite M200 Microplate Reader (Tecan Deutschland, Germany) at 230 nm.

Adsorption Isotherms with Proteins

0.5 mL protein solution was mixed with a magnetite particle solution 1 g L⁻¹ in a 1.5 mL Eppendorf vessel and incubated for 1 h at 25° C. and 1000 rpm in a thermomixer comfort from Eppendorf.

The particles and the supernatant were separated with a NdFeB magnet. The supernatant was centrifuged at 17000×g at 4° C. for 5 minutes in order to remove residual particles prior to analysis.

### High-Gradient Magnetic Separation

The process starts with the incubation of 0.5 L cell lysate in TBS pH 7 containing Glu₆-GFP with 0.5 L magnetite (2 g/L) in a feed tank. This mixture is stirred at −900 rpm for 1 h at room temperature. Every 15 min aliquots of 2 mL are taken in order to monitor the adsorption.

After the incubation, the suspension is pumped through a magnetic separation chamber as described (Roth et al. A High-Gradient Magnetic Separator for Highly Viscous Process Liquors in Industrial Biotechnology, Chemical Engineering and Technology 39 (3) 469-476). The supernatant containing impurities and unbound protein is removed by two washing steps, each 0.5 L containing TBS pH 7. The flow direction through the chamber is changed every 200 s during the washing procedure. During the washing procedure 10 samples are taken of the outlet fractions. For the elution citrate buffered saline (CBS) at pH 7 is used which is provided in the feed tank. The particles are resuspended as the magnetic field is removed and the whole mixture is circulated through the whole apparatus with changing flow directions every 15 minutes. Aliquots are taken each 10 minutes and after 1 h the suspension is pumped through the magnetic field again in order to separate the protein from the particles. In a second step the resuspension is improved by the addition of a two phase flow where air is mixed with the buffer for 10 minutes as described (Roth et al. A High-Gradient Magnetic Separator for Highly Viscous Process Liquors in Industrial Biotechnology, Chemical Engineering and Technology 39 (3) 469-476). Aside from the two phase flow through the system, the process is similar to the first elution step.

### EXAMPLE 2

### Peptide Arrays

The binding behavior of magnetite nanoparticles (MNPs) to peptides was investigated using a peptide array. FIG. 1 shows that at a pH of 7.4 in Tris buffered saline, the negatively charged peptides hexa-glutamic acid (6E) and hexa-aspartic acid (6D) bound strongest to the MNPs. The second highest scores at pH 7.4 were achieved by the positive peptides hexa-arginine (6R), hexa-lysine (6K), 5RH and 5RE as well as hexa-histidine (6H) which is nearly neutral at a pH of 7.4 according to the theoretical pl of 7.21 determined by the ProtParam Tool by the Bioinformatics Resource Portal ExPASy (Bjellqvist, B., et al. (1993): The focusing positions of polypeptides in immobilized pH gradients can be predicted from their amino acid sequences, Electrophoresis 14 (1), pp. 1023-1031). It is in accordance with literature that histidine residues can form complexes with metals and that carboxy groups bind them through electrostatic interaction (Kozlowski, H., et al. (2013): Specific metal ion binding sites in unstructured regions of proteins, Coordination Chemistry Reviews 257 (19-20), pp. 2625-2638). The MNP surface was shown to be slightly positively charged in zetapotential measurements at a pH of 7.4 as shown in Table 1. The point of zero charge was determined to be around 7.8 by acidometry. Hydrophobic peptides do not show any binding while the binding of polar peptides is low to moderate.

TABLE 1
Zetapotential of magnetic nanoparticles
in different buffers and at different pHs

| Buffer | pH | Zetapotential, mV |
|---|---|---|
| T-TBS | 7.4 | +3.7 |
| | 8 | |
| T-PBS | 6 | −27.8 |
| | 7.4 | |
| | 8 | |
| T-CBS | 6 | −34.74 |

### EXAMPLE 3

Comparison of Homooligomers with Heterooligomers

As demonstrated in FIG. 7, heteromeric peptides with interspersed serine, glycine or glutamine residues exhibit better binding than homooligomers of glutamine or asparagines. The heteromers have a length between 9 and 12 amino acids and the fraction of residues which are negatively charged at neutral pH is at least ⅔.

As shown in FIG. 8, such results could be confirmed on a different array format. In particular, to the extent the fraction of cysteine, glycine or asparagines is above ⅓, binding drops below the binding of the corresponding homooligomers. On the other hand, if the fraction of cysteine, glycine or asparagines does not exceed ⅓, binding is increased as compared to the corresponding homooligomers.

FIG. 9 shows affinity constants of peptides in accordance with the present invention to magnetic nanoparticles in different buffers and at different pH values.

### EXAMPLE 4

### Separation Method

The data depicted in FIG. 10 demonstrate that peptides in accordance with the present invention when used in proteins to be purified allow binding to magnetic nanoparticles. In a tris buffered system, Glu₆-, Glu₆-, and Glu₄Gly₄Glu₄-tagged protein binds with high affinity and high maximal loads as compared to untagged protein; see also Table 2 below.

TABLE 2
Binding constants of tagged proteins to MNPs in T-TBS at pH 7.

| Protein | qₘₐₓ, g · g⁻¹ | K_{D}, g · L⁻¹ |
|---|---|---|
| GFP-Glu₈ | 0.179 ± 0.006 | 4E−04 ± 2E−04 |
| GFP-Glu₆ | 0.120 ± 0.008 | 4E−15 ± 3E−03 |
| GFP-E₄G₄E₄ | 0.102 ± 0.011 | 7E−10 ± 6E−03 |
| GFP-Gly₆ | 0.047 ± 0.005 | 1E−03 ± 1E−03 |
| GFP no tag | 8E−05 ± 1E−03 | 2E−16 ± 6E−01 |

Using a magnetic separator (as described in Roth et al., Chem. Eng. Technol. 2016, 39, 469-476) has been used to separate GFP tagged with a peptide in accordance with the invention (Gluₓ) from a bacterial broth using magnetic particles in TBS. CBS has been used as elution buffer. FIG. 11 displays the concentration of the protein of interest (GFP-Glu₆) and the total protein concentration as a function of time.

1. A peptide consisting of a sequence of 5 to 30 amino acids, wherein
(a) at least ⅔ of said amino acids have a functional group or side chain which is negatively charged at neutral pH;
(b) amino acids which do not have a functional group or side chain which is negatively charged at neutral pH, if present, meet one or both of requirements (i) and (ii):
(i) none of them has a functional group or side chain which is positively charged at neutral pH; and
(ii) at least one of them has a side chain which does not bear a net charge at neutral pH or which has a functional group or side chain that does not bear a net charge at neutral pH.

2. The peptide of claim 1, wherein the fraction of amino acids which have a functional group or side chain which is positively charged at neutral pH is
(a) in the range between zero and 0.2; or
(b) greater than 0.2.

3. The peptide of claim 1 or 2, wherein said at least one amino acid as defined in claim 1(b)(ii) is present and is flanked on either side by at least one amino acid as defined in claim 1(a).

4. The peptide of claim 1, wherein said peptide is capable of binding to a surface comprising iron oxide.

5. The peptide of claim 1, wherein
(a) at least one amino acid has a side chain which is negatively charged at neutral pH are selected from Asp and Glu; and/or
(b) at least one amino acid, to the extent present, which has a side chain which does not bear a net charge at neutral pH are selected from Gly, Ser, Ala, Asn, Cys, Gln, His, Ile, Leu, Met, Phe, Pro, Thr, Trp, Tyr, Val and selenocystein.

6. A molecule which is a polypeptide or protein, said polypeptide or protein comprising at least one sequence of a peptide as defined in any one of the preceding claims, wherein the sequence(s) in said polypeptide or protein which do(es) not comprise said at least one sequence of a peptide are heterologous with respect to said sequence of a peptide.

7. A molecule covalently bound to at least one sequence of a peptide as defined in claim 1, wherein the covalent bond is not a main chain peptide bond, and wherein said molecule is a polypeptide or protein or a nucleic acid.

8. A surface comprising iron oxide, preferably a magnetic nanoparticle, which is bound to at least one peptide or molecule as defined in claim 1.

9. A method of separating a molecule as defined in claim 6 from a sample, said method comprising:
(a) contacting said sample with a surface comprising iron oxide, preferably magnetic nanoparticles, under conditions allowing binding of said molecule to said surface; and
(b) separating said surface from said sample;
thereby separating said molecule from said sample.

10. A method of separating an analyte from a sample, said analyte being capable of binding to a molecule as defined in claim 6, said method comprising:
(a) bringing into contact said sample, said molecule and a surface comprising iron oxide, preferably magnetic nanoparticles, under conditions allowing (i) binding of said molecule to said surface and (ii) binding of said analyte to said molecule; and
(b) separating said surface from said sample;
thereby separating said analyte from said sample.

11. The method of claim 9 or 10, further comprising
(c) washing said surface; and/or
(d) (i) dissociating said molecule from said surface;
(ii) optionally followed by removing said surface from the result of (i).

12. The method of claim 11, wherein said dissociating in step (d)(i) is performed by adding
(a) a carboxylic acid, preferably a carboxylic acid comprising two or three carboxylic groups; and/or
(b) an inorganic oxo acid.

13. A method of determining presence or absence of an analyte in a sample suspected of comprising said analyte, said method comprising
(a) bringing into contact said sample, said molecule and a surface comprising iron oxide, under conditions allowing (i) binding of said molecule to said surface and (ii) binding of said analyte to said molecule, and
(b) separating said surface from said sample,
thereby separating said analyte from said sample; and
optionally at least one of steps (c), (d)(i), (d)(ii) and (e) as defined in claim 11, thereby obtaining a mixture, and (f) analyzing said mixture for the presence of said analyte.

14. A kit comprising
(a) (i) a peptide as defined claim 1;
(ii) a molecule which is a polypeptide or protein, wherein the sequence(s) in said polypeptide or protein do(es) not comprise said at least one sequence of a peptide are heterologous with respect to said sequence of the peptide as defined in claim 1; and/or
(iii) a nucleic acid encoding a peptide of (i) or a molecule of (ii), wherein said molecule is a polypeptide or protein.

15. The kit of claim 14, further comprising at least one of (b), (c), (d) and (e):
(b) a surface comprising iron oxide;
(c) a first solution or constituents for preparing said first solution, said first solution being capable of establishing conditions which allow binding of said peptide and/or said molecule to said surface;
(d) a second solution or constituents for preparing said second solution, said second solution being capable of establishing conditions which allow dissociating said peptide and/or said molecule from said surface; and
(e) instructions for use of said kit, instructing separating a molecule which is a polypeptide or protein, said polypeptide or protein comprising at least one sequence of a peptide as defined in any one of the preceding claims,
wherein the sequence(s) in said polypeptide or protein which do(es) not comprise said at least one sequence of a peptide are heterologous with respect to said sequence of a peptide from a sample, said method comprising:
(i) contacting said sample with a surface comprising iron oxide, preferably magnetic nanoparticles, under conditions allowing binding of said molecule to said surface; and
(ii) separating said surface from said sample.


SEQUENCE LISTING (19 sequences)

SEQ ID NO: 1 - protein, 6 aa, Artificial Sequence
  iron oxide-binding peptide
EEEEEE

SEQ ID NO: 2 - protein, 6 aa, Artificial Sequence
  iron oxide-binding peptide
DDDDDD

SEQ ID NO: 3 - protein, 12 aa, Artificial Sequence
  iron oxide-binding peptide
DDDDEDDEDDDD

SEQ ID NO: 4 - protein, 12 aa, Artificial Sequence
  iron oxide-binding peptide
DDDEDDEDDEDD

SEQ ID NO: 5 - protein, 10 aa, Artificial Sequence
  iron oxide-binding peptide
DDDDDDDDDD

SEQ ID NO: 6 - protein, 12 aa, Artificial Sequence
  iron oxide-binding peptide
DDDDDEDDDDDD

SEQ ID NO: 7 - protein, 10 aa, Artificial Sequence
  iron oxide-binding peptide
DDDEDDEDDD

SEQ ID NO: 8 - protein, 12 aa, Artificial Sequence
  iron oxide-binding peptide
DDDDDDDDDDDD

SEQ ID NO: 9 - protein, 10 aa, Artificial Sequence
  iron oxide-binding peptide
DDEDDEDDED

SEQ ID NO: 10 - protein, 10 aa, Artificial Sequence
  iron oxide-binding peptide
DDDDEDDDDD

SEQ ID NO: 11 - protein, 12 aa, Artificial Sequence
  iron oxide-binding peptide
DDDDDGGDDDDD

SEQ ID NO: 12 - protein, 8 aa, Artificial Sequence
  iron oxide-binding peptide
DDDDDDDD

SEQ ID NO: 13 - protein, 10 aa, Artificial Sequence
  iron oxide-binding peptide
DDDDGGDDDD

SEQ ID NO: 14 - protein, 12 aa, Artificial Sequence
  iron oxide-binding peptide
DDDDGGGGDDDD

SEQ ID NO: 15 - protein, 10 aa, Artificial Sequence
  iron oxide-binding peptide
DDDGGGGDDD

SEQ ID NO: 16 - protein, 8 aa, Artificial Sequence
  iron oxide-binding peptide
DDDGGDDD

SEQ ID NO: 17 - protein, 12 aa, Artificial Sequence
  iron oxide-binding peptide
DDDGGGGGGDDD

SEQ ID NO: 18 - protein, 11 aa, Artificial Sequence
  iron oxide-binding peptide
DDDDDDDDDDD

SEQ ID NO: 19 - protein, 12 aa, Artificial Sequence
  iron oxide-binding peptide
EEEEGGGGEEEE